(19) United States
(12) Patent Application Publication
(10) Pub. No.: US 2011/0318795 A1
(43) Pub. Date: Dec. 29, 2011

(54) DICARBOXYLIC ACID PRODUCTION IN A FILAMENTOUS FUNGUS

(76) Inventors: René Verwaal, Nootdorp (NL); Liang Wu, Delft (NL); Robbertus Antonius Damveld, Berkel en Rodenrijs (NL); Cornelis Maria Jacobus Sagt, Utrecht (NL)

(21) Appl. No.: 12/743,416

(22) PCT Filed: Nov. 14, 2008
(86) PCT No.: PCT/EP08/65582

§ 371 Date: May 18, 2010

(30) Foreign Application Priority Data
Nov. 20, 2007 (EP) 07121111.4
May 27, 2008 (EP) 08156958.4

(51) Int. Cl. C12N 1/15; C12P 7/46; C12P 7/44

(52) U.S. Cl. 435/145; 435/254.11; 435/254.3; 435/254.5; 435/254.9; 435/142

(57) ABSTRACT

The present invention relates to a recombinant fungus comprising an enzyme which catalyses the conversion of malic acid to fumaric acid in the cytosol. The invention further relates to a process for the production of a dicarboxylic acid such as fumaric acid and succinic acid, wherein the recombinant fungus is used.

The present invention relates to a recombinant filamentous fungus, comprising an enzyme which catalyses the conversion of malic acid to fumaric acid in the cytosol, and a process for the production of a dicarboxylic acid.

Dicarboxylic acids such as fumaric acid and succinic acid are potential precursors for numerous chemicals. For example, succinic acid can be converted into 1,4-butanediol (BDO), tetrahydrofuran, and gamma-butyrolactone. Another product derived from succinic acid is a polyester polymer which is made by linking succinic acid and BDO.

Succinic acid is predominantly produced through petrochemical processes by hydrogenation of butane. These processes are considered harmful for the environment and costly. The fermentative production of succinic acid may be an attractive alternative process for the production of succinic acid, wherein renewable feedstock as a carbon source may be used.

A number of different bacteria such as Escherichia coli, and the rumen bacteria Actinobacillus, Anaerobiospirillum, Bacteroides, Mannheimia, or Succinimonas, sp. are known to produce succinic acid. Metabolic engineering of these bacterial strains have improved the succinic acid yield and/or productivity, or reduced the by-product formation.

WO2007/061590 discloses a pyruvate decarboxylase negative yeast for the production of malic acid and/or succinic acid which is transformed with a pyruvate carboxylase enzyme or a phosphoenolpyruvate carboxylase, a malate dehydrogenase enzyme, and a malic acid transporter protein (MAE).

Despite the improvements that have been made in the fermentative production of dicarboxylic acids, there remains a need for improved microorganisms for the fermentative production of dicarboxylic acids.

The aim of the present invention is an alternative microorganism for the production of dicarboxylic acids.

The aim is achieved according to the present invention by a recombinant filamentous fungus, comprising a nucleotide sequence encoding an enzyme which catalyses the conversion of malic acid to fumaric acid in the cytosol, wherein the enzyme has at least 70% identity to SEQ ID NO: 1 or SEQ ID NO: 3, preferably to SEQ ID NO: 3. Surprisingly, it was found that the recombinant filamentous fungus according to the present invention produces a higher amount of dicarboxylic acids, such as fumaric acid and succinic acid, compared to the wild type filamentous fungus. Preferably, the amount of fumaric acid and/or succinic acid produced by a recombinant filamentous fungus according to the present invention is at least 2, preferably at least 2.5, 3, 4, or 5 times higher than the amount produced by a wild type filamentous fungus.

Usually, the enzyme catalysing the conversion of malic acid to fumaric acid is active in the cytosol upon expression of the nucleotide sequence.

As used herein, a recombinant fungus according to the present invention is defined as a fungus which contains, or is transformed or genetically modified with a nucleotide sequence that does not naturally occur in (a compartment of) the fungus, or it contains additional copy or copies of an endogenous nucleic acid sequence. A wild-type fungus is the parental fungus of the recombinant fungus.

The nucleotide sequence encoding an enzyme which catalyses the conversion of malic acid to fumaric acid in the cytosol may be a heterologous or homologous nucleotide sequence, which may be derived from any suitable origin, for instance bacteria, fungi, protozoa or plants. Preferably the nucleotide sequence encodes a fumarase. Preferably, the enzyme that catalyses the conversion of malic acid to fumaric acid in the cytosol, is a homologous enzyme derived from the filamentous fungus according to the present invention.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but have been obtained from another cell or synthetically or recombinantly produced.

Preferably, the fungus according to the present invention is a filamentous fungus comprising a nucleotide sequence encoding an enzyme which catalyses the conversion of malic acid to fumaric acid in the cytosol, wherein the enzyme has at least 75% identity, preferably at least 80, 85, 90, 92, 94, 95, 96, 97, 98, 99% identity to SEQ ID NO: 1 or SEQ ID NO: 3, preferably to SEQ ID NO: 3.

Sequence identity is herein defined as a relationship between two or more amino acid (polypeptide or protein) sequences or two or more nucleic acid (polynucleotide) sequences, as determined by comparing the sequences. Usually, sequence identities are compared over the whole length of the sequences compared. In the art, “identity” also means the degree of sequence relatedness between amino acid or nucleic acid sequences, as the case may be, as determined by the match between strings of such sequences.

Preferred methods to determine identity are designed to give the largest match between the sequences tested. Methods to determine identity are codified in publicly available computer programs. Preferred computer program methods to determine identity between two sequences include BLASTP and BLASTN publicly available from NCBI and other sources (BLAST Manual, Altschul, S., et al., NCBI NLM NIH Bethesda, Md. 20894). Preferred parameters for amino acid sequences comparison using BLASTP are gap open 11.0, gap extend 1, Blosum 62 matrix.

Nucleotide sequences encoding an enzyme which catalyses the conversion of malic acid to fumaric acid in the cytosol according to the invention may also be defined by their capability to hybridise with the nucleotide sequences encoding an enzyme of SEQ ID NO: 1 or SEQ ID NO: 3, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC (sodium chloride, sodium citrate) or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequence of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

To increase the likelihood that the enzyme according to the present invention is expressed in active form in the fungus of the invention, the corresponding encoding nucleotide sequence may be adapted to optimise its codon usage to that of the chosen eukaryote host cell. Several methods for codon optimisation are known in the art. A preferred method to optimise codon usage of the nucleotide sequences to that of the fungus is a codon pair optimization technology as disclosed in WO2008/000632. Codon-pair optimization is a method for producing a polypeptide in a host cell, wherein the nucleotide sequences encoding the polypeptide have been modified with respect to their codon-usage, in particular the codon-pairs that are used, to obtain improved expression of the nucleotide sequence encoding the polypeptide and/or improved production of the polypeptide. Codon pairs are defined as a set of two subsequent triplets (codons) in a coding sequence.

The term “gene”, as used herein, refers to a nucleic acid sequence containing a template for a nucleic acid polymerase, in eukaryotes, RNA polymerase II. Genes are transcribed into mRNAs that are then translated into protein.

The term “nucleic acid” as used herein, includes reference to a deoxyribonucleotide or ribonucleotide polymer, i.e. a polynucleotide, in either single-or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). A polynucleotide can be full-length or a subsequence of a native or heterologous structural or regulatory gene. Unless otherwise indicated, the term includes reference to the specified sequence as well as the complementary sequence thereof.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation.

The term “enzyme” as used herein is defined as a protein which catalyses a (bio)chemical reaction in a cell, such as in a cell.

Usually, a nucleotide sequence encoding an enzyme, for instance an enzyme that catalyses the conversion of malic acid to fumaric acid, is operably linked to a promoter that causes sufficient expression of the corresponding nucleotide sequence in the fungus according to the present invention.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences known to one of skilled in the art. A “constitutive” promoter is a promoter that is active under most environmental and developmental conditions. An “inducible” promoter is a promoter that is active under environmental or developmental regulation.

The promoter that could be used to achieve the expression of a nucleotide sequence coding for an enzyme according to the present invention, may be not native to the nucleotide sequence coding for the enzyme to be expressed, i.e. a promoter that is heterologous to the nucleotide sequence (coding sequence) to which it is operably linked. Preferably, the promoter is homologous, i.e. endogenous to the host cell.

Suitable promoters in this context include both constitutive and inducible natural promoters as well as engineered promoters, which are well known to the person skilled in the art. Suitable promoters in eukaryotic host cells may be GAL7, GAL10, or GAL 1, CYC1, HIS3, ADH1, PGL, PH05, GAPDH, ADC1, TRP1, URA3, LEU2, ENO, TPI, and AOX1. Other suitable promoters include PDC, GPD1, PGK1, TEF1, and TDH.

Usually a nucleotide sequence encoding an enzyme comprises a terminator. Any terminator, which is functional in the eukaryotic cell, may be used in the present invention. Preferred terminators are obtained from natural genes of the host cell. Suitable terminator sequences are well known in the art. Preferably, such terminators are combined with mutations that prevent nonsense mediated mRNA decay in the host cell of the invention (see for example: Shirley et al., 2002, Genetics 161:1465-1482).

In a preferred embodiment, the nucleotide sequence encoding an enzyme which catalyses the conversion of malic acid to fumaric acid is overexpressed to achieve an increased production of a dicarboxylic acid, such as fumaric acid and/or succinic acid by the fungus according to the present invention.

There are various means available in the art for overexpression of enzymes in the eukaryotic cell of the invention. In particular, an enzyme may be overexpressed by increasing the copy number of the gene coding for the enzyme in the cell, e.g. by integrating additional copies of the gene in the cell's genome, by expressing the gene from a centromeric vector, from an episomal multicopy expression vector or by introducing an (episomal) expression vector that comprises multiple copies of the gene. Preferably, overexpression of the enzyme according to the invention is achieved with a (strong) constitutive promoter, for instance a GPDA promotor.

The invention also relates to a nucleotide construct comprising one or more nucleotide sequence(s) of SEQ ID NO: 4.

The nucleic acid construct may be a plasmid, for instance a low copy plasmid or a high copy plasmid. The fungus according to the present invention may comprise a single, but preferably comprises multiple copies of the nucleotide sequence encoding an enzyme that catalyses the conversion of malic acid to fumaric acid, for instance by multiple copies of a nucleotide construct.

The nucleic acid construct may be maintained episomally and thus comprise a sequence for autonomous replication, such as an autosomal replication sequence. A suitable episomal nucleic acid construct may e.g. be based on the yeast 2μ or pKD1 plasmids (Gleer et al., 1991, Biotechnology 9: 968-975), or the AMA plasmids (Fierro et al., 1995, Curr Genet. 29:482-489). Alternatively, each nucleic acid construct may be integrated in one or more copies into the genome of the fungus. Integration into the cell's genome may occur at random by non-homologous recombination but preferably, the nucleic acid construct may be integrated into the cell's genome by homologous recombination as is well known in the art (see e.g. WO90/14423, EP-A-0481008, EP-A-0635 574 and U.S. Pat. No. 6,265,186).

It was found that the nucleotide sequence encoding an enzyme that catalyses the conversion of malic acid to succinic acid, may comprise a peroxisomal or mitochondrial targeting signal. It was found preferred to modify or delete a number of amino acids comprising a peroxisomal or mitochondrial targeting signal (and corresponding nucleotide sequences in the encoding nucleotide sequence) to target the enzyme to the cytosol. The presence of a peroxisomal targeting signal may for instance be determined by the method disclosed by Schluter et al, Nucleic acid Research 2007, Bol 25, D815-D822. Preferably, the enzyme that catalyses the conversion from malic acid to fumaric acid is a truncated form of a homologous enzyme of the filamentous fungus according to the present invention. A truncated enzyme as used herein is an enzyme that does not comprise a mitochondrial or peroxisomal targeting signal.

The filamentous fungus preferably belongs to one of the genera Aspergillus, Penicillium, or Rhizopus. More preferably, the filamentous fungus is a Aspergillus niger, Aspergillus oryzae, a Penicillium chrysogenum, or Rhizopus oryzae.

In addition to a nucleotide sequence encoding an enzyme that catalyses the conversion of malic acid to fumaric acid, the recombinant filamentous fungus according to the present invention may comprise further genetic modifications, for instance mutations, deletions or disruptions, in homologous nucleotide sequences and/or transformation with nucleotide sequences that encode homologous or heterologous enzymes that catalyse a reaction in the cell resulting in an increased flux towards fumaric acid and/or succinic acid. It may for example be favourable to introduce, genetically modify and/or overexpress heterologous and/or homologous nucleotide sequences encoding i) an enzyme that catalyses the conversion of phosphoenolpyruvate or pyruvate to oxaloacetate; ii) a malate dehydrogenase which catalyses the conversion from OAA to malic acid; or iii) a fumarase, which catalyses the conversion of malic acid to fumaric acid.

The fungus may be transformed or genetically modified with any suitable nucleotide sequence catalyzing the reaction from a C3 to C4 carbon molecule, such as phosphoenolpyruvate (PEP, C3) to oxaloacetate (OAA,C4) and pyruvate (C3) to OAA or malic acid. Suitable enzymes are PEP carboxykinase (EC 4.1.1.49, EC 4.1.1.38) and PEP carboxylase (EC 4.1.1.31) which catalyse the conversion of PEP to OAA; pyruvate carboxylase (EC 6.4.1.1.), that catalyses the reaction from pyruvate to OAA; or malic enzyme (EC 1.1.1.38), that catalyses the reaction from pyruvate to malic acid.

Preferably, the enzymes under i), ii) and iii) are expressed in the cytosol. Cytosolic expression may be achieved by deletion or modification of a mitochondrial or peroxisomal targeting signal as has been described herein before. Further molecular DNA techniques as described herein above, such as overexpression and codon optimization are also applicable to these nucleotide sequences/proteins.

In another preferred embodiment the recombinant filamentous fungus according to the present invention comprises at least one gene encoding succinate dehydrogenase that is not functional. A succinate dehydrogenase that is not functional is used herein to describe a fungus, which comprises a reduced succinate dehydrogenase activity by mutation, disruption, or deletion, of at least one gene encoding succinate dehydrogenase, resulting in an increased formation of succinic acid as compared to the wild-type cell. A fungus comprising a gene encoding succinate dehydrogenase that is not functional may for instance be Aspergillus niger, preferably an Aspergillus niger, wherein one or more genes encoding succinate dehydrogenase, such as sdhA is not functional, for instance by deletion of the gene. Surprisingly, it was found that the succinic acid production increased in a filamentous fungus wherein a succinate dehydrogenase was not functional.

Preferably, the filamentous fungus according to the present invention is an A. niger comprising one or more nucleotide sequences of SEQ ID NO: 4.

A preferred filamentous fungus according to the present invention may be able to grow on any suitable carbon source known in the art and convert it to a dicarboxylic acid, such as fumaric acid and/or succinic acid. The fungus may be able to convert directly plant biomass, celluloses, hemicelluloses, pectines, rhamnose, galactose, fucose, maltose, maltodextrines, ribose, ribulose, or starch, starch derivatives, sucrose, lactose and glycerol. Hence, a preferred filamentous fungus expresses enzymes such as cellulases (endocellulases and exocellulases) and hemicellulases (e.g. endo- and exo-xylanases, arabinases) necessary for the conversion of cellulose into glucose monomers and hemicellulose into xylose and arabinose monomers, pectinases able to convert pectines into glucuronic acid and galacturonic acid or amylases to convert starch into glucose monomers. Preferably, the cell is able to convert a carbon source selected from the group consisting of glucose, fructose, galactose, xylose, arabinose, sucrose, lactose, raffinose, and glycerol into a dicarboxylic acid.

In another aspect, the present invention relates to a process for the preparation of a dicarboxylic acid such as fumaric acid or succinic acid, comprising fermenting a filamentous fungus according to the present invention in a suitable fermentation medium.

It was found advantageous to use a filamentous fungus according to the invention in the process for the production of a dicarboxylic acid, such as fumaric acid and/or succinic acid, because this resulted in an increased production of fumaric acid and/or succinic acid as compared to the use of a wild-type filamentous fungus.

Preferably, the dicarboxylic acid produced in the process according to the present invention is succinic acid.

The process according to the present invention may be run under aerobic and anaerobic conditions. Preferably, the process is carried out under micro-aerophilic or oxygen limited conditions.

An anaerobic fermentation process is herein defined as a fermentation process run in the absence of oxygen or in which substantially no oxygen is consumed, preferably less than 5, 2.5 or 1 mmol/L/h, and wherein organic molecules serve as both electron donor and electron acceptors.

An oxygen-limited fermentation process is a process in which the oxygen consumption is limited by the oxygen transfer from the gas to the liquid. The degree of oxygen limitation is determined by the amount and composition of the ingoing gasflow as well as the actual mixing/mass transfer properties of the fermentation equipment used. Preferably, in a process under oxygen-limited conditions, the rate of oxygen consumption is at least about 5.5, more preferably at least about 6 and even more preferably at least about 7 mmol/L/h.

The process for the production of a dicarboxylic acid, such as fumaric acid and/or succinic acid according to the present invention may be carried out at any suitable pH between 1 and 9. Preferably, the pH in the fermentation broth is between 2 and 7, preferably between 3 and 5. It was found advantageous to be able to carry out the process according to the present invention at a low pH, since this prevents bacterial contamination. In addition, since the pH drops during succinic acid production, a lower amount of titrant may be needed to keep the pH at a desired (low) level.

A suitable temperature at which the process according to the present invention may be carried out is between 5 and 60° C., preferably between 10 and 50° C., more preferably between 15 and 35° C., more preferably between 18° C. and 30° C. The skilled man in the art knows which optimal temperatures are suitable for fermenting a specific filamentous fungus.

Preferably, the dicarboxylic acid, such as fumaric acid and/or succinic acid, is recovered from the fermentation broth by a suitable method known in the art, for instance by crystallisation and ammonium precipitation.

Preferably, the dicarboxylic acid that is prepared in the process according to the present invention is further converted into a pharmaceutical, cosmetic, food, feed, or chemical product. Succinic acid may be further converted into a polymer, such as polybutylene succinate (PBS) or other suitable polymers derived therefrom

In another embodiment the present invention relates to the use of dicarboxylic acid produced according to a process according to the present invention for the preparation of a pharmaceutical, cosmetic, food, feed or chemical product.

Genetic Modifications

Standard genetic techniques, such as overexpression of enzymes in the host cells, genetic modification of host cells, or hybridisation techniques, are known methods in the art, such as described in Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation, genetic modification etc of fungal host cells are known from e.g. EP-A-0 635 574, WO 98/46772, WO 99/60102 and WO 00/37671, WO90/14423, EP-A-0481008, EP-A-0635 574 and U.S. Pat. No. 6,265,186.

The following examples are for illustrative purposes only and are not to be construed as limiting the invention.

### DESCRIPTION OF THE FIGURES

FIG. 1. Map of the pGBTOP-11 vector used for expression of fumarase in A. niger.

FIG. 2: Map of plasmid pGBTOPAn3 for FUMm overexpression in A. niger.

FIG. 3: Plasmid map of pDEL-SDHA

FIG. 4: Replacement scheme of sdhA

### EXAMPLES

### Example 1

### Overexpression of Aspergillus niger Fumarase in Aspergillus niger

### 1.1. Expression Constructs

Fumarase [E.C. 4.2.1.2], GenBank accession number 145247174 from Aspergillus was analysed for the presence of signal sequences using SignalP 3.0 (http://www.cbs.dtu.dk/services/SignalP/) Bendtsen, J. et al. (2004) Mol. Biol., 340:783-795 and TargetP 1.1 (http://www.cbs.dtu.dk/services/TargetP/) Emanuelsson, O. et al. (2007) Nature Protocols 2, 953-971.

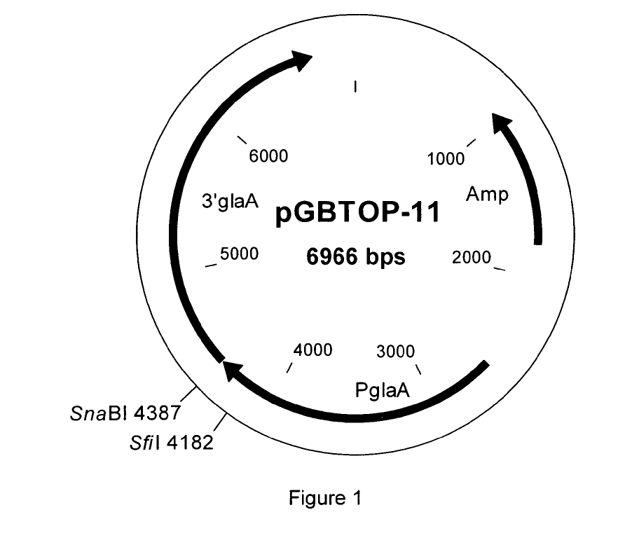

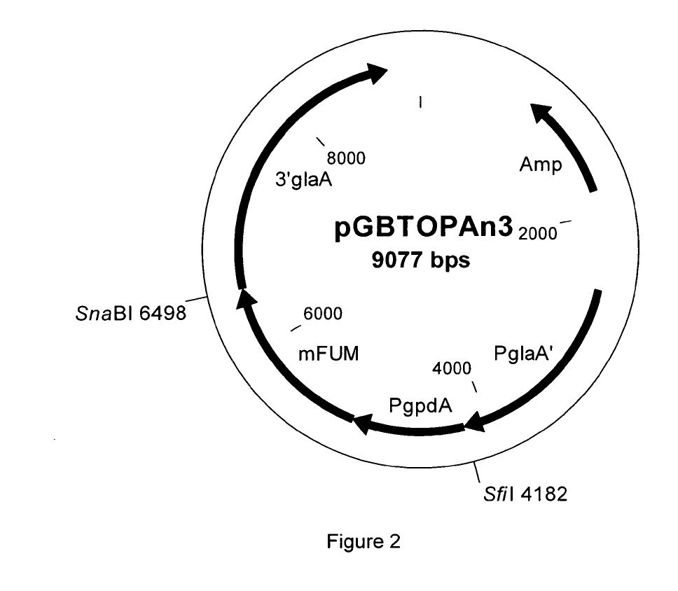

A putative mitochondrial targeting sequence in the first 67 amino acid of the protein was identified. To avoid potential targeting to mitochondria in A. niger, the first 67 amino acids were removed from SEQ ID NO: 1 (corresponding nucleotide sequence SEQ ID NO: 2), and a methionine amino acid was reintroduced at the start, resulting in SEQ ID NO: 3. SEQ ID NO: 4 was put behind the constitutive GPDA promoter (source pGBAAS-1) sequence SEQ ID NO: 5, wherein the last 10 nucleotide sequences were replaced with optimal Kozak sequence (CACCGTAAA). The stop codon TAA in SEQ ID NO: 4 was modified into TAAA. Convenient restriction sites were added. The resulting sequence was synthesised at Sloning (Puchheim, Germany). The fragment was SnaBI, SfiI cloned in the A. niger expression vector pGBTOP11 (see FIG. 1) using appropriate restriction sites. The resulting plasmid comprising SEQ ID NO: 4, was renamed pGBTOPAn3 (FIG. 2)

1.2. Transformation of A. niger

A. niger WT-1: This A. niger strain is CBS513.88 comprising deletions of the genes encoding glucoamylase (glaA), fungal amylase and acid amylase. A. niger WT 1 is constructed by using the “MARKER-GENE FREE” approach as described in EP 0 635 574 B1.

The expression construct comprising fumarase is co-transformed to strain A. niger WT-1 according to the method described by Tilburn, J. et al. (1983) Gene 26, 205-221 and Kelly, J. & Hynes, M. (1985) EMBO J., 4, 475-479 with the following modifications:
Spores are germinated and cultivated for 16 hours at 30 degrees Celsius in a shake flask placed in a rotary shaker at 300 rpm in Aspergillus minimal medium (100 ml). Aspergillus minimal medium contains per litre: 6 g NaNO₃, 0.52 g KCl, 1.52 g KH₂PO₄, 1.12 ml 4 M KOH, 0.52 g MgSO₄.7H₂O, 10 g glucose, 1 g casaminoacids, 22 mg ZnSO₄.7H₂O, 11 mg H₃BO₃, 5 mg FeSO₄.7H₂O, 1.7 mg CoCl₂.6H₂O, 1.6 mg CuSO₄.5H₂O, 5 mg MnCl₂.2H₂O, 1.5 mg Na₂MoO₄.2H₂O, 50 mg EDTA, 2 mg riboflavin, 2 mg thiamine-HCl, 2 mg nicotinamide, 1 mg pyridoxine-HCL, 0.2 mg panthotenic acid, 4 g biotin, 10 ml Penicillin (5000 IU/ml) Streptomycin (5000 UG/ml) solution (Gibco).
Novozym 234™ (Novo Industries) instead of helicase is used for the preparation of protoplasts;
After protoplast formation (60-90 minutes), KC buffer (0.8 M KCl, 9.5 mM citric acid, pH 6.2) is added to a final volume of 45 ml, the protoplast suspension is centrifuged for 10 minutes at 3000 rpm at 4 degrees Celsius in a swinging-bucket rotor. The protoplasts are resuspended in 20 ml KC buffer and subsequently 25 ml of STC buffer (1.2 M sorbitol, 10 mM Tris-HCl pH 7.5, 50 mM CaCl₂) is added. The protoplast suspension is centrifuged for 10 minutes at 3000 rpm at 4 degrees Celsius in a swinging-bucket rotor, washed in STC-buffer and resuspended in STC-buffer at a concentration of 10E8 protoplasts/ml;
To 200 microliter of the protoplast suspension, the DNA fragment, dissolved in 10 microliter TE buffer (10 mM Tris-HCl pH 7.5, 0.1 mM EDTA) and 100 microliter of PEG solution (20% PEG 4000 (Merck), 0.8 M sorbitol, 10 mM Tris-HCl pH 7.5, 50 mM CaCl₂) is added;
After incubation of the DNA-protoplast suspension for 10 minutes at room temperature, 1.5 ml PEG solution (60% PEG 4000 (Merck), 10 mM Tris-HCl pH 7.5, 50 mM CaCl₂) is added slowly, with repeated mixing of the tubes. After incubation for 20 minutes at room temperature, suspensions are diluted with 5 ml 1.2 M sorbitol, mixed by inversion and centrifuged for 10 minutes at 4000 rpm at room temperature. The protoplasts are resuspended gently in 1 ml 1.2 M sorbitol and plated onto solid selective regeneration medium consisting of either Aspergillus minimal medium without riboflavin, thiamine.HCL, nicotinamide, pyridoxine, panthotenic acid, biotin, casaminoacids and glucose. In case of acetamide selection the medium contains 10 mM acetamide as the sole nitrogen source and 1 M sucrose as osmoticum and C-source. Alternatively, protoplasts are plated onto PDA (Potato Dextrose Agar, Oxoid) supplemented with 1-50 microgram/ml phleomycin and 1M sucrose as osmosticum. Regeneration plates are solidified using 2% agar (agar No. 1, Oxoid L11). After incubation for 6-10 days at 30 degrees Celsius, conidiospores of transformants are transferred to plates consisting of Aspergillus selective medium (minimal medium containing acetamide as sole nitogen source in the case of acetamide selection or PDA supplemented with 1-50 microgram/ml phleomycin in the case of phleomycin selection) with 2% glucose and 1.5% agarose (Invitrogen) and incubated for 5-10 days at 30 degrees Celsius. Single transformants are isolated and this selective purification step is repeated once upon which purified transformants are stored.

1.3. Shake Flask Growth of A. niger

In total 10 transformants are selected for each construct and the presence of the construct is confirmed by PCR using primers specific for the constructs. Subsequently spores are inoculated in 100 ml Aspergillus minimal enriched medium comprising 100 g/l glucose. Strains are grown in an incubator at 250 rotations per minute for four days at 34 degrees Celsius. The supernatant of the culture medium is analysed for oxalic acid, malic acid, fumaric acid and succinic acid formation by HPLC and compared to a non transformed strain.

1.4 HPLC Analysis

HPLC is performed for the determination of organic acids and sugars in different kinds of samples. The principle of the separation on a Phenomenex Rezex-RHM-Monosaccharide column is based on size exclusion, ion-exclusion and ion-exchange using reversed phase mechanisms. Detection takes place by differential refractive index and ultra violet detectors.

### Example 2

### Inactivation of Succinate Dehydrogenase Encoding Genes in Aspergillus niger

2.1. Identification

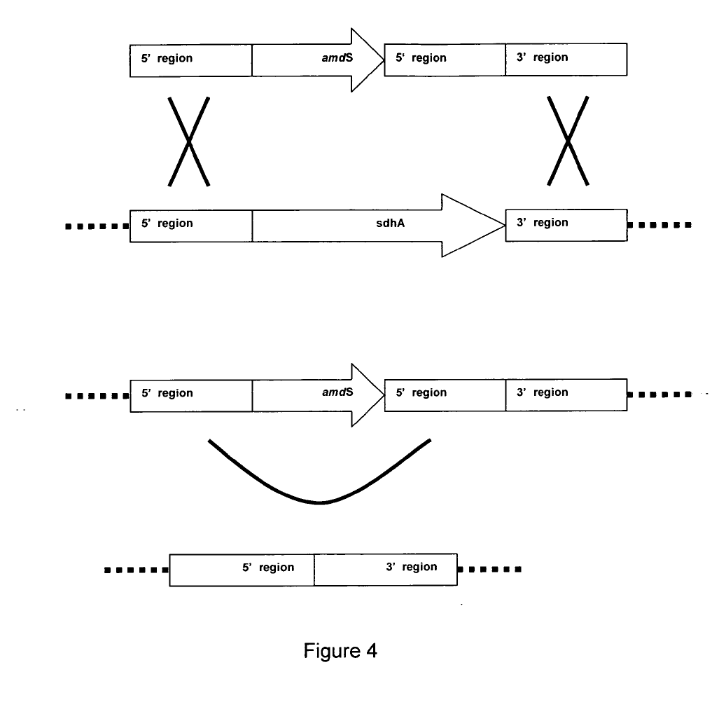

Genomic DNA of Aspergillus niger strain CBS513.88 was sequenced and analyzed. Two genes with translated proteins annotated as homologues to succinate dehydrogenase proteins were identified and named sdhA and sdhB, respectively. Sequences of the sdhA (An16g07150) and sdhB (An02g12770) loci are available on genbank with accession numbers 145253004 and 145234071, respectively. Gene replacement vectors for sdhA were designed according to known principles and constructed according to routine cloning procedures (see FIG. 4). The vectors comprise approximately 1000 bp flanking regions of the sdh ORFs for homologous recombination at the predestined genomic loci. In addition, they contain the A. nidulans bi-directional amdS selection marker driven by the gpdA promoter, in-between direct repeats. The general design of these deletion vectors were previously described in EP635574B and WO 98/46772.

2.2. Inactivation of the sdhA Gene in Aspergillus niger.

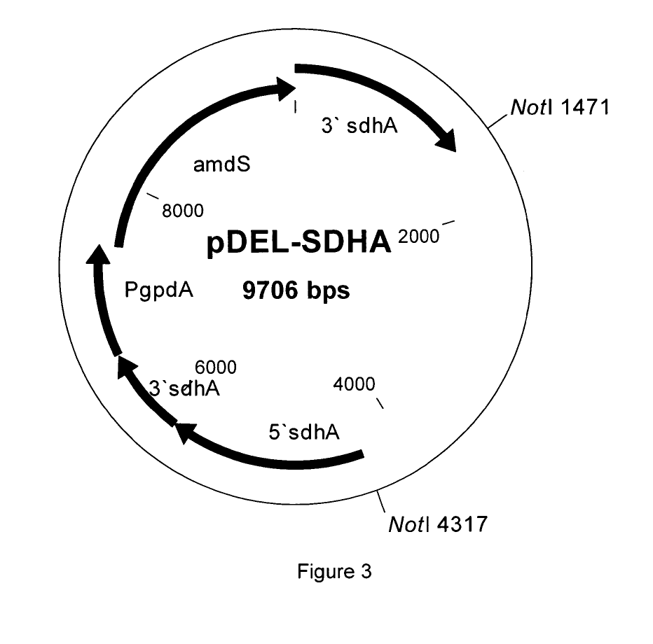

Linear DNA of deletion vector pDEL-SDHA (FIG. 3) was isolated and used to transform Aspergillus niger CBS513.88 according to the methods as described in: Biotechnology of Filamentous fungi: Technology and Products. (1992) Reed Publishing (USA); Chapter 6: Transformation p. 113 to 156. This linear DNA can integrate into the genome at the sdhA locus, thus substituting the sdhA gene by the amdS gene as depicted in FIG. 4. Transformants were selected on acetamide media and colony purified according to standard procedures as described in EP635574B. Spores were plated on fluoro-acetamide media to select strains, which lost the amdS marker. Growing colonies were diagnosed by PCR for integration at the sdhA locus and candidate strains tested by Southern analyses for deletion of the sdhA gene. Deletion of the sdhA gene was detectable by the ˜2.2 kb size reduction of DNA fragments (4.6 kb wild-type fragment versus 2.4 kb for a succesfull deletion of SDHA) covering the entire locus and hybridized to appropriate probes. Approximately 9 strains showed a removal of the genomic sdhA gene from a pool of approximately 96 initial transformants.

Strain dSDHA was selected as a representative strain wherein the sdhA gene is inactivated. Strain dSDHA was cultivated in microtiter plates as described below and the amount of succinic acid produced was measured by HPLC as described in Example 1.4.

### Example 3

### Fumarase Overexpression in an A. niger dSDHA

A. niger strain dSDHA of example 2.2. was transformed with the expression construct pGBTOPAn3 (FIG. 2) comprising truncated fumarase (SEQ ID NO: 4) as described in example 1.1. E. coli DNA was removed by NotI digestion. The transformation procedure followed was as described in example 1.2. A. niger transformants were picked using Qpix and transferred onto microtiter plates (MTP) containing Aspergillus selective media. After 7 days of growth at 30 degrees Celsius, biomass was transferred to microtiter plates (MTP's) containing PDA. After 7 days of incubation at 30 degrees Celsius, the biomass was sporulated. These spores were resuspended using the Multimek 96 (Beckman) in 100 microlitres minimal enriched Aspergillus medium (see Example 1.2) containing 10% glucose. Subsequently, 2 MTP's with 170 microlitres minimal enriched Aspergillus medium containing 10% glucose and 1% CaCO3 were inoculated with 30 microlitres of the spore suspension. Likewise, A. niger strains dSDHA and CBS513.88 were inoculated the MTP's. These MTP's were incubated for 5 days at 34 degrees, 550 rpm at 80% humidity. After 5 days 160 microlitres were harvested using the Multimek 96 (Beckman) and analysed for succinic acid by HPLC as described in example 1.4. The results are shown in Table 1.

TABLE 1
Effect of a deletion succinate dehydrogenase (SDHA) and cytosolic
activity of fumarase in A. niger in the production of succinic acid.

| A. niger strain | Succinic acid (g/l) |
|---|---|
| CBS513.88 | 0.038 |
| dSDHA | 0.05 |
| dSDHA + pGBTOPAn3 (FumM) | 0.268 |

The results in Table 1 show that deletion of succinate dehydrogenase and expression of fumarase in the cytosol of A. niger increases the succinic acid production levels.

1. A recombinant filamentous fungus comprising a nucleotide sequence encoding an enzyme which catalyses the conversion of malic acid to fumaric acid in the cytosol, wherein the enzyme has at least 70% identity to SEQ ID NO: 3.

2. A fungus according to claim 1, wherein the fungus is of the genus Aspergillus, Penicillium or Rhizopus.

3. A fungus according to claim 1, wherein the fungus is of the species Aspergillus niger, Aspergillus oryzae, Penicillium chrysogenum, or Rhizopus oryzae.

4. A fungus according to claim 1, wherein the enzyme is overexpressed.

5. A fungus according to claim 1, wherein the enzyme is a fumarase.

6. A fungus according to claim 1, wherein the enzyme is a truncated form of a homologous enzyme.

7. A fungus according to claim 1, wherein at least one gene encoding succinate dehydrogenase is not functional.

8. A fungus according to claim 1, wherein the fungus is of the species Aspergillus niger comprising SEQ ID NO: 4.

9. A process for the preparation of a dicarboxylic acid, comprising fermenting the filamentous fungus according to claim 1 in a suitable fermentation medium, wherein a dicarboxylic acid is produced

10. Process according to claim 9 wherein a dicarboxylic acid is further converted into a pharmaceutical, cosmetic, food, feed or chemical product.

11. Process for the production of a dicarboxylic acid selected from fumaric acid and succinic acid using a filamentous fungus as dicarboxylic acid producer, whereby fumarase is used to increase dicarboxylic acid production.

12. Process according to claim 11, wherein the increase is at least two times.

13. Process according to claim 11, wherein the fumarase is active in the cytosol.


SEQUENCE LISTING (5 sequences)

SEQ ID NO: 1 - protein, 533 aa, Aspergillus niger
MASLTHAASRASASPAVARTAVAFTPASFSCRRLLSSNSRPVQHFPRLQTLTSTSSKRAFGTTVKMSSATRIETDAFGEIEVPADKYWGAQTQRSLGNFDINQPQDRMPEPVVKAFGILKGAAAEVNMKFGLDPKIGEAIKQAAAEVAEGKLMDHFPLVVWQTGSGTQSNMNSNEVISNRAIEILGGEKGSKKPVHPNDHVNMSASSNDSFPTAMHIAAVVELENTLLPSLRSLRDALQVKVEKFDKIIKIGRTHLQDATPLTLGQEFSGYVAQLDRNIERVETSIPHLRYLAQGGTAVGTGLNTFKGFDEAIAAEVTKLTGTEFKTAPNKFEVLAAHDSIVEASGALNTLACSLFKIAQDIRYLGSGPRCGLGELVLPENEPGSSIMPGKVNPTQCESLTMVCSQVMGNHVAATVGGMNGQFELNVFKPLMIRNLLHSVRILADGMASFEKNLVHGLEANEPRINSLLHESLMLVTCLNPVIGYDMASKVAKNAHKKGLTLKQSAMELKALSEEDFDKYVRPELMLSPKEKK

SEQ ID NO: 2 - DNA, 1601 bp, Aspergillus niger
atggcctccttgacacatgctgcatctcgagcttcggcttcccccgcagttgcccgcactgccgtcgctttcacccccgcctcgttcagttgccgtcgtcttctgagctccaatagccgacctgttcaacacttcccccgtcttcagaccttgacttccacctccagcaagagagcctttggcaccaccgtcaagatgtcttcggctacccgcattgagaccgatgccttcggtgagatcgaggtccccgccgacaagtactggggtgcccagacccagcgctccctgggcaacttcgacatcaaccagccccaggaccgcatgcctgagcccgttgtcaaggctttcggtatcctcaagggtgctgctgctgaagtgaacatgaagttcggccttgaccccaagatcggcgaggccatcaagcaggctgccgccgaggttgcggagggcaagctgatggaccacttccccctcgtcgtctggcagaccggttccggtacccagtccaacatgaactcgaacgaggtcatctccaaccgcgccattgagatcctgggcggcgagaagggctccaagaagcccgtccaccccaacgaccacgtcaacatgtccgcctcctccaatgactccttcccgaccgctatgcacattgctgccgttgtggagctggagaacaccctcctgccttccctgaggagcctgcgcgatgctctccaggtcaaggttgagaagttcgacaagatcatcaagatcggtcgtactcacctgcaggacgccacccctctcaccctcggtcaggagttctccggctacgtcgctcagctcgaccgcaacattgagcgtgtcgagactagcatcccccacctccgctacctggctcagggtggtaccgccgtcggtactggtctgaacaccttcaagggcttcgacgaggctatcgctgctgaggtcaccaagttgaccggcaccgagttcaagactgcccccaacaagttcgaggttctggccgcccacgactcgattgtcgaggcttccggtgccctgaacaccctggcctgctctctgttcaagattgcccaggacatccgttaccttggatccggtccccgctgcggtcttggtgaactggtcctccccgagaacgagcctggctcttccatcatgcccggcaaggttaaccccactcagtgcgagtcccttaccatggtctgctcccaggtcatgggtaaccacgtcgctgccactgtcggcggcatgaacggtcagttcgagctcaacgtgttcaagcccctcatgatccgcaacctgctgcacagcgtgcgcatcctggccgatggcatggccagcttcgagaagaacctggtgcacggtctggaggccaacgagccccgcatcaactctctcctccacgagagtctgatgttggtcacctgcctgaaccccgtcattggctacgacatggcctccaaggtcgccaagaacgcccacaagaagggcctcactctgaagcagagtgctatggagctgaaggctctgagcgaggaggatttgacaagtacgtccgcccggagctgatgctgagccccaaggagaagaaataa

SEQ ID NO: 3 - protein, 467 aa, Artificial sequence
  mFUM A. niger minus 67 aa targeting signal
MSATRIETDAFGEIEVPADKYWGAQTQRSLGNFDINQPQDRMPEPVVKAFGILKGAAAEVNMKFGLDPKIGEAIKQAAAEVAEGKLMDHFPLVVWQTGSGTQSNMNSNEVISNRAIEILGGEKGSKKPVHPNDHVNMSASSNDSFPTAMHIAAVVELENTLLPSLRSLRDALQVKVEKFDKIIKIGRTHLQDATPLTLGQEFSGYVAQLDRNIERVETSIPHLRYLAQGGTAVGTGLNTFKGFDEAIAAEVTKLTGTEFKTAPNKFEVLAAHDSIVEASGALNTLACSLFKIAQDIRYLGSGPRCGLGELVLPENEPGSSIMPGKVNPTQCESLTMVCSQVMGNHVAATVGGMNGQFELNVFKPLMIRNLLHSVRILADGMASFEKNLVHGLEANEPRINSLLHESLMLVTCLNPVIGYDMASKVAKNAHKKGLTLKQSAMELKALSEEDFDKYVRPELMLSPKEKK

SEQ ID NO: 4 - DNA, 1404 bp, Artificial sequence
  nt mFUM minus putative targeting signal
atgtcggctacccgcattgagactgatgccttcggtgagatcgaggtccccgccgacaagtactggggtgcccagacccagcgctccctgggcaacttcgacatcaaccagccccaggaccgcatgcctgagcccgttgtcaaggctttcggtatcctcaagggtgctgctgctgaagtgaacatgaagttcggccttgaccccaagatcggcgaggccatcaagcaggctgccgccgaggttgcggagggcaagctgatggaccacttccccctcgtcgtctggcagaccggttccggtacccagtccaacatgaactcgaacgaggtcatctccaaccgcgccattgagatcctgggcggcgagaagggctccaagaagcccgtccaccccaacgaccacgtcaacatgtccgcctcctccaatgactccttcccgaccgctatgcacattgctgccgttgtggagctggagaacaccctcctgccttccctgaggagcctgcgcgatgctctccaggtcaaggttgagaagttcgacaagatcatcaagatcggtcgtactcacctgcaggacgccacccctctcaccctcggtcaggagttctccggctacgtcgctcagctcgaccgcaacattgagcgtgtcgagactagcatcccccacctccgctacctggctcagggtggtaccgccgtcggtactggtctgaacaccttcaagggcttcgacgaggctatcgctgctgaggtcaccaagttgaccggcaccgagttcaagactgcccccaacaagttcgaggttctggccgcccacgactcgattgtcgaggcttccggtgccctgaacaccctggcctgctctctgttcaagattgcccaggacatccgttaccttggatccggtccccgctgcggtcttggtgaactggtcctccccgagaacgagcctggttcttccatcatgcccggcaaggttaaccccactcagtgcgagtcccttaccatggtctgctcccaggtcatgggtaaccacgtcgctgccactgtcggcggcatgaacggtcagttcgagctcaacgtgttcaagcccctcatgatccgcaacctgctgcacagcgtgcgcatcctggccgatggcatggccagcttcgagaagaacctggtgcacggtctggaggccaacgagccccgcatcaactctctcctccacgagagtctgatgttggtcacctgcctgaaccccgtcattggctacgacatggcctccaaggtcgccaagaacgcccacaagaagggcctcactctgaagcagagtgctatggagctgaaggctctgagcgaggaggactttgacaagtacgtccgcccggagctgatgctgagccccaaggagaagaaataa

SEQ ID NO: 5 - DNA, 898 bp, Artificial sequence
  GPDA promotor
tcagcgtccaattcgagctctgtacagtgaccggtgactctttctggcatgcggagacacggacggtcgcagagaggagggctgagtaataagcgcactcatgtcagctctggcgctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagccaccgtcaaa